(19) United States
(12) Patent Application Publication
(10) Pub. No.: US 2007/0032801 A1
(43) Pub. Date: Feb. 8, 2007

(54) Vascular suturing device

(76) Inventors: Anthony J. Pantages, San Jose, CA (US); Brian Ellingwood, Sunnyvale, CA (US); Erik Walberg, Redwood City, CA (US)

Correspondence Address: WORKMAN NYDEGGER, 1000 EAGLE GATE TOWER,, 60 EAST SOUTH TEMPLE, SALT LAKE CITY, UT, 84111, US

(21) Appl. No.: 11/199,338

(22) Filed: Aug. 8, 2005

(51) Int. Cl. A61B 17/04

(52) U.S. Cl. 606/148

(57) ABSTRACT

A surgical device of suturing vascular vessels is described, as well as methods for suturing tissue employing the surgical device. The device includes at least one needle advanceable through tissue to drawn lengths of suture material which can then be used to close various puncture wounds, particularly in vascular tissue. A foot is pivotal between a non-deployed position and a deployed position where it engages vascular tissue on a distal side of the vessel.

### BACKGROUND OF THE INVENTION

The present invention generally relates to surgical instruments and methods of suturing tissue.

A number of diagnostic and treatment procedures are conducted intravascularly. Typically, a catheter is introduced into the vascular system at a convenient access location and is then guided to the target treatment site. The Seldinger Technique is one of the well-known early examples of this type of procedure which can include catheterization and angioplasty techniques. Procedures such as this require a vascular access. Typically an introducer sheath with or without a guide wire is inserted through a puncture wound in a vessel such as the femoral artery at a location near the groin. A catheter and other instrumentation can then be inserted through the sheath and guided to the targeted treatment site. After the diagnostic and/or treatment procedure has been completed, the puncture wound must be closed. Closing the wound can be difficult because of the substantial bleeding that can occur through an open wound in the vascular vessel. One technique for hemostasis includes applying pressure near or upstream of the puncture site. This approach suffers from many deleterious effects, not the least of which are that it can be time consuming and extremely uncomfortable—even painful—for the patient because the pressure is applied directly on or adjacent to the traumatized site. Frequently anticoagulants are employed for the original diagnostic/treatment procedures. This delays clot formation during the procedure, and this effect lasts through the initial recovery period, lengthening the time during which pressure must be applied to the wound for up to twelve hours or more. During this initial recovery period, it is imperative that the patient remain still, further adding to the patient's discomfort.

Alternatively, the puncture wound can be closed with sutures. This can be extremely difficult because the vascular vessel with the puncture lies underneath the patient's outer skin. Some vascular vessels, notably the femoral artery, appear to be relatively large; however, in practice, even the largest arteries cannot be readily sutured. Therefore, devices have been developed to facilitate subcutaneous suturing of arteries and veins. These devices can extend through the outer tissue to the puncture wound in the vascular vessel. Needles are then deployed from the device to suture the tissue adjacent the puncture wound.

Certain devices are inserted through the wound and initially deploy needles to pierce the tissue in from outside the vascular vessel and continue on into a depository in the portion of the device located within the lumen of the vessel. The suturing device can be removed from the vessel (and the patient) by withdrawing the needles and suture material at the same time. These devices leave an inverted suture path after completion of the closure. The suture material runs from the exterior tissue surrounding the puncture wound back up through the wound itself which is then tied off. Some complications may arise resulting from this type of closure, including oozing, excessive bleeding, and, on rare occasions, knot loosening. It would be preferred to provide a suturing device that allows the suture path to extend across the puncture opening internal of the vessel membrane with the suture knot overlying the exterior of the closed wound.

Furthermore, while the above-described techniques are regularly performed, as with any surgical procedure, they involve considerable risk to patients. These particular procedures entail delicate and intricate procedures. The physical condition of the patient and, importantly, the condition of the patient's vascular system can greatly impact the risks and prognosis. For example, a patient's vascular vessels may be more or less compliant. This can cause further complications making suturing of the puncture wound more difficult. Many current suturing devices cannot accommodate and support non-compliant vascular tissue because the sutures can be misplaced, not attach sufficient tissue, or pull out making wound closure more risky for these patients. Devices which can support tissue and facilitate correct suture placement would reduce some of the risks attendant with this procedure.

In view of the above background, there remain needs for improved and/or alternative methods and devices for closing vascular opening or punctures. The present invention is addressed to these needs.

### SUMMARY OF THE INVENTION

The present invention relates to suturing device and the use thereof. Various aspects of the invention are novel, nonobvious, and provide various advantages. While the actual nature of the invention covered herein can only be determined with reference to the claims appended hereto, certain forms and features, which are characteristic of the preferred embodiments disclosed herein, are described briefly as follows.

In one form the present invention provides a suturing device for suturing an opening in a vascular vessel. The device comprises: a proximal member including an elongate body with a needle channel therethrough sized to receive at least one needle; a distal member configured to be inserted within a lumen of a vascular vessel, where the distal member has a receptacle to receive one or more of the needles after located they have passed through the tissue; an intermediate member disposed between the proximal member and the distal member and defining a tissue receiving area with a first opening providing a passageway to the channel and a second opening providing a passageway into the receptacle; and a foot pivotally mounted on the intermediate member to engage with a portion of the tissue.

In another form the present invention provides a method of suturing an opening in a vascular vessel. The method comprises: inserting a suturing device through the opening in the vascular vessel wherein the suturing device comprises a proximal member having a needle channel therein; a distal member configured to be inserted into the lumen of the vascular vessel with a receptacle therein sized to receive at least one needle; an intermediate member disposed between the proximal and distal member and having a first opening providing a passageway way to the needle channel and a second opening providing a second passageway into the receptacle and wherein the intermediate member is angled or curved to offset the needle channel and the receptacle from the opening in the vascular vessel; and a foot pivotally mounted on the intermediate member. The method also comprises: deploying the foot to contact vascular tissue adjacent the opening; and advancing a first needle through the needle channel to pierce a portion of vascular tissue at a first suture site adjacent the opening in the vascular vessel and into the receptacle in the distal member. Additionally the device can be relocated within the vessel to advance a second needle and length of suture material through the tissue. The ends of the two lengths of suture material can be pulled taut to close the wound or opening the vessel. A surgical knot or other knot replacement technology can be utilized to complete the wound closure.

### BRIEF DESCRIPTION OF THE DRAWINGS

FIG. 1 is a perspective view of one embodiment of a suturing device with an articulating foot according to the present invention.

FIG. 2 is an enlarged fragmentary view of the intermediate member of the suturing device illustrated in FIG. 1.

FIG. 3 is a plan view of the suturing device illustrated in FIG. 1.

FIG. 4 is a cross-section view taken on line 4-4 of the suturing device illustrated in FIG. 3.

FIG. 5 is a longitudinal cross-sectional view along section 5-5 of the suturing device illustrated in FIG. 4 illustrating the intermediate member with the articulating foot in a non-deployed position.

FIG. 6 is a fragmentary cross-section view of the suturing device illustrated in FIG. 1 with the foot in a first, non-deployed position.

FIG. 7 is a fragmentary cross-section view of the suturing device illustrated in FIG. 1 illustrating the intermediate member with the articulating foot in a second or deployed position.

FIGS. 8-11 illustrate use of the suturing device of FIG. 1.

FIG. 12 is a perspective view of one embodiment of a suture securing device for use in the present invention.

FIG. 13 is a perspective view of an alterative embodiment of a suture securing device for use in the present invention.

FIG. 14 is a prospective view of one embodiment of a suturing device with a needle capture element in accordance with the present invention.

FIG. 15 is an exploded view of one embodiment of a suturing device including a length of suture material with fitting for engaging a needle in accordance with the present invention.

FIG. 16 is a cross-section view of a fitting and a needle for use with the suturing device in accordance with the present invention.

### DETAILED DESCRIPTION OF THE INVENTION

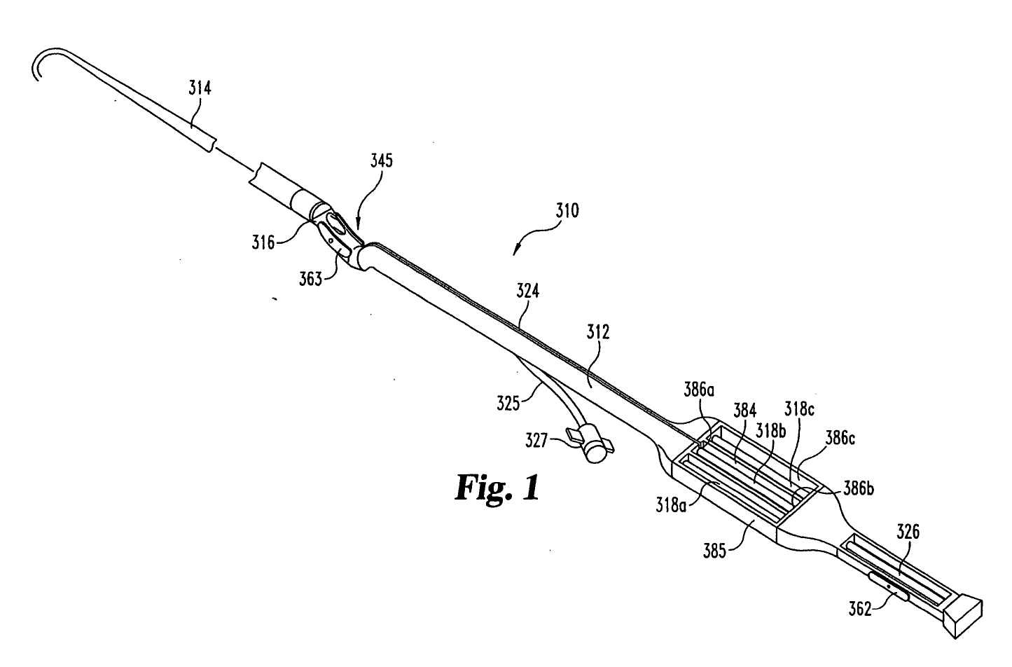

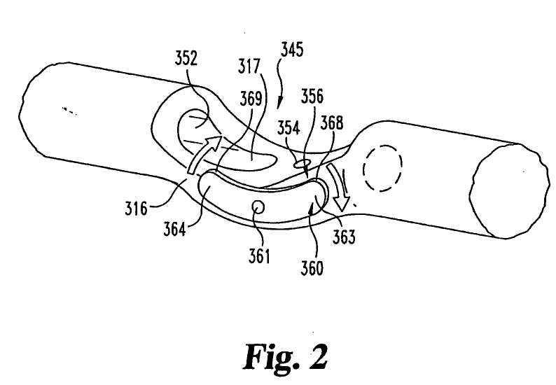

FIGS. 1 and 2 illustrate one embodiment of a suturing device 310 for use in the present invention. Suturing device 310 includes a proximal member 312, a distal member 314, and an intermediate member 316 disposed therebetween. Proximal member 312 is provided as an elongated portion and can exhibit a substantially cylindrical or oval radial cross section. Member 312 includes a first end of sufficient dimensions to be readily grasped by the surgeon to manipulate the device during the procedures. Proximal member 312 can also include a gripping portion to facilitate handling during the surgical procedure. A needle channel 324 runs longitudinally along at least a portion of proximal member. In one embodiment, channel 324 extends along the entire length of proximal member from a first end positioned proximal to the surgeon to a second end adjacent to intermediate member 316. In this embodiment, one or more needle(s) 318 and needle pusher(s) 326 can be inserted into and retrieved from channel 324 at the first end. In other embodiments, channel 324 extends only partly through the proximal member 312. Needle channel 324 can be centrally located along proximal member 312. In preferred embodiments, proximal member 312 includes a single needle channel 324 through which one, two, three, or more needles can be advanced. Alleviating multiple needle channels within the suturing device provides a more compact member, which can be particularly advantageous for subcutaneous procedures.

As used herein, the term “proximal” refers to a direction toward the surgeon and away from the patient or a location closer to the surgeon, while the term “distal” refers to a direction towards the patient and away from the surgeon or a location closer to the patient.

Channel 324 is sized and dimensioned to allow one or more needles 318 to be advanceable therethrough and into vascular tissue around the puncture wound. Furthermore, channel 324 can be either partly or completely encased within the body of proximal member 312. However, in a preferred embodiment, channel 324 is not encased within the body of proximal member 312. Rather, channel 324 is provided as a slot formed into the surface of proximal member 312. Preferably the slot is configured to retain one or more needles within the slot. For example, the slot can be formed to have an opening at the exterior surface of proximal member that is narrower than the diameter of the needles (and, optionally, the pusher) while the internal portion or diameter of the slot can be dimensioned to permit facile movement of the needle therethrough. An exit opening is located at the distal end of channel 324.

Proximal member 312 includes a blood return line 325 that terminates in a fitting 327, for example, a luer lock that can be mated to a syringe. Alternatively, line 325 can terminate in a valve or shunt to control and stop blood flow therethrough. It is preferable that blood line 325 allow visible observation of blood originating from inside the vascular vessel. This can facilitate proper placement of the device for suturing.

Suturing device 310 comprises a needle cartridge 384 slidably mounted in a body 385 disposed on proximal member 312. Needle cartridge 384 can include two, three, four or more needle slots 386a, 386b, and 386c . . . for needles 318a, 318b, 318c , . . . Each of needles 318a, 318b, 318c, in needle cartridge 384 is individually advanceable through a central needle channel 324 along a length of proximal member 312. Needle cartridge 384 is slidably disposed within body 385 to axially align the selected needle slot 386a, 386b, 386c with needle channel 324. If desired, needle cartridge 384 can be biased to automatically align the successive needle slots with channel 324 after the preceding needle has been advanced. Alternatively, body 385 and/or cartridge 384 can include one or more of ratchetings, positive stops, or locks to align the desired needle slot with channel 324. In other embodiments, needle cartridge 384 can be provided as a revolving barrel that can hold two, three, or more needles in respective needle slots radially disposed about the barrel. The barrel can be rotatably mounted on or about proximal member 312.

A needle pusher 326 can either push or engage needle 318 to advance it through a channel 324 in the proximal member and through vascular tissue adjacent the puncture wound. In a preferred embodiment, needle pusher 326 in conjunction with needle cartridge can be configured such that the surgeon can sequentially advance needles 318a, 318b, and 318c , . . . in a proximal direction towards the patient.

Each of needles 318a, 318b, 318c , . . . can be attached to a length of suture material prior to be being loaded into the needle cartridge. The length of suture material can be attached adjacent to either the distal end of the proximal end. Preferably, the length of suture material is attached adjacent to the proximal end. In another embodiment, a single length of suture material is attached at one end to a first needle and at a second end to a second needle. The distal end of the needle can be configured to pierce tissue such as vascular tissue. The distal end can taper to a point, be configured as a barbed tip or include recessed surfaces to engage in either fittings or the needle capture elements. Examples of needles with recessed surfaces are illustrated and described in copending U.S. patent application No. ______, filed on Aug. 8, 2005 (and listed under Attorney Docket No. 7323US01), examples of needles suitable for use with a needle capture element are illustrated and described in copending U.S. patent application No. ______, filed on Aug. 8, 2005 (and listed under Attorney Docket No. 7968US01), each which is incorporated by reference in their entirety herein. The proximal end 34 of needle can be free and configured to be handled by a surgeon. Alternatively, proximal end can be engageable or secured to needle pusher 326. In a preferred embodiment, needle pusher 326 is configured such that the surgeon can sequentially advance needle 318 in a proximal direction towards the patient and in a distal direction away from the patient.

Suturing device 310 includes distal member 314. In certain embodiments, distal member 314 and/or proximal member 312 is/are linear and define a longitudinal axis. Distal member 314 is sized and/or configured to be received within an opening or wound to a lumen of a patient's vascular vessel. Therefore, it is preferable that at least distal member 314 be formed of a flexible or elastomeric material that is biocompatible—particularly with blood. In additional embodiments, distal member 314 can be coated or impregnated with a lubricant, bioactive agent, such as an anticoagulant material, and the like. In certain embodiments, distal member 314 is composed of a biocompatible polymeric material commonly used for catheters, such as silicone rubber, polyolefin polyurethane, polytetrafluoroethylene, and the like.

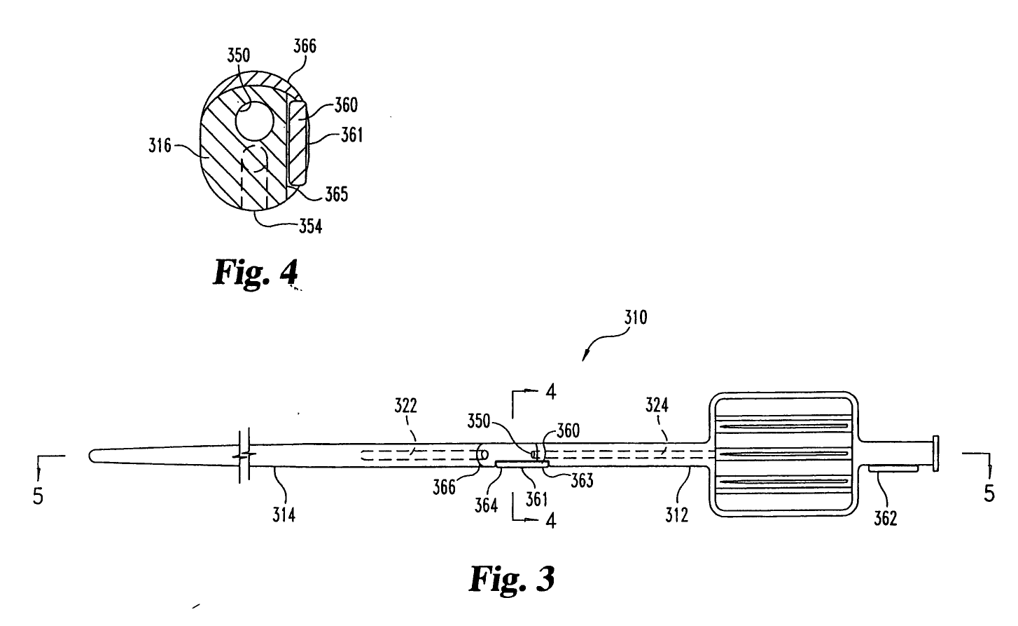

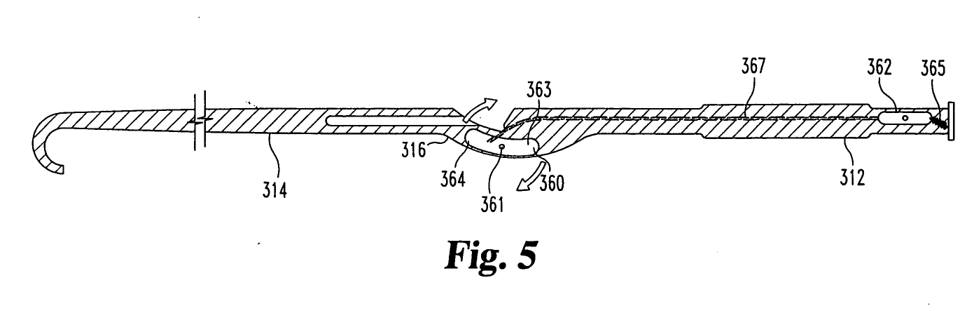

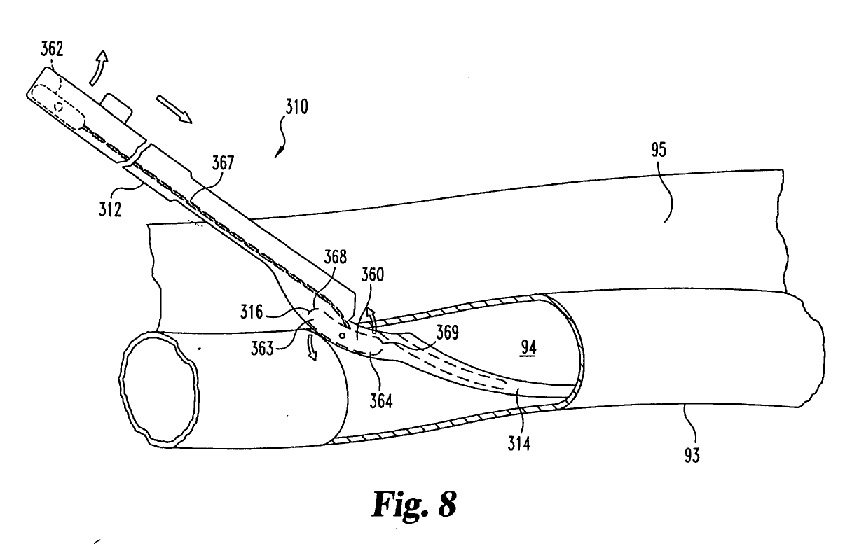

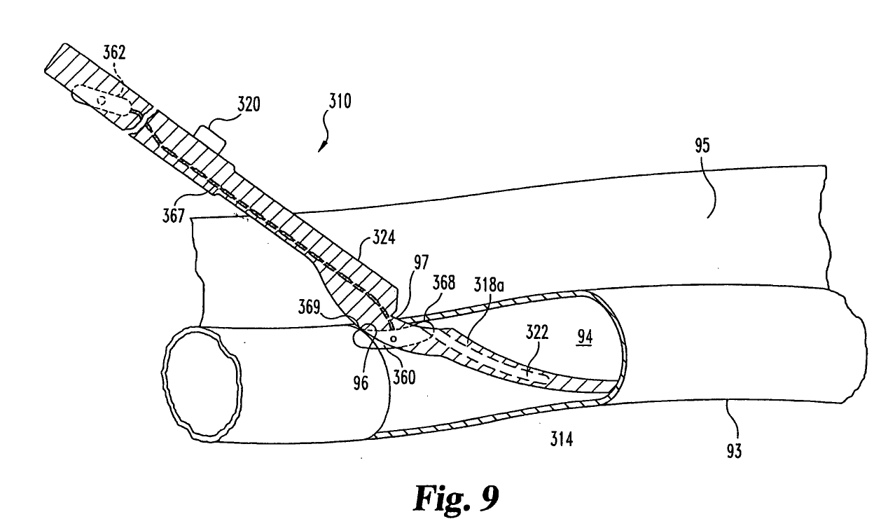

Referring specifically to FIGS. 2 and 3, intermediate member 316 is positioned between proximal member 312 and distal member 314. Intermediate member 316 is configured to include an arcuate portion or a crooked section 317. An articulating foot 360 is pivotally secured to intermediate member 316 via pivot pin 361. Preferably, foot 360 is adjacent to the tissue receiving area 345 defined by intermediate member 316. Lever 362 located axially on proximal member 312 operates to position foot 360 between a first, non-deployed position to a second, deployed position back again to the first, non-deployed position (see FIGS. 6 and 7). In certain embodiments, foot 360 is symmetric about pivot pin 361 extending in both the proximal direction and the distal direction the about the same length—measured from the center of pivot pin 361. In other embodiments, foot 360 is asymmetric in the longitudinal direction in that either the proximal end 363 or the distal end 364 extends further from pivot pin 361 than the other end. It will also be understood that proximal end 363 can, but need not, be the mirror image of distal end 364. (See FIG. 6.) Intermediate member 316 also includes means and structure for reliable positioning of the device during surgery to facilitate closing the vascular puncture wound with sutures. Part of the positioning structure includes an opening 354 providing fluid communication to blood return line 325 in proximal member 312. In a preferred embodiment, opening 354 (see FIG. 4) is located on a portion of the convex surface of the crook opposite the tissue receiving region. In use, with the distal member of the device suitably positioned within the lumen of a vascular vessel, opening 354 is also located in the interior of the lumen. This permits blood from the vessel to enter blood return line 325, which can then be visibly observed by the surgeon. If blood is not observed in blood return line 325, then the distal member may not have been inserted to a sufficient depth into the lumen of the vascular vessel.

Additionally, a ridge or stop 356 extends from the concave surface into the tissue receiving region. Stop 356 is configured to bear against vascular tissue adjacent the puncture wound. In a preferred embodiment, first opening 350 extends through a portion of stop 356 permitting needle 318 to pierce tissue adjacent thereto. Stop 356 is sized to bear against the vascular tissue and avert further insertion of the device 310 into the vascular vessel. When provided together, stop 356 and opening 354 (with blood return line 325) cooperate to ensure accurate placement of the suturing device in the patient's vascular vessel. Ridge or stop 356 can also extend radially about the entire circumference of intermediate member 316.

Referring now to FIGS. 3 and 4, foot 360 is illustrated as nesting within a depression 365 formed in the lateral surface 366 of intermediate member 316. In this embodiment, foot 360 is configured to provide or complete the circular or oval radial external profile of member 316 to facilitate insertion and use of the device to suture wounds in vascular vessel.

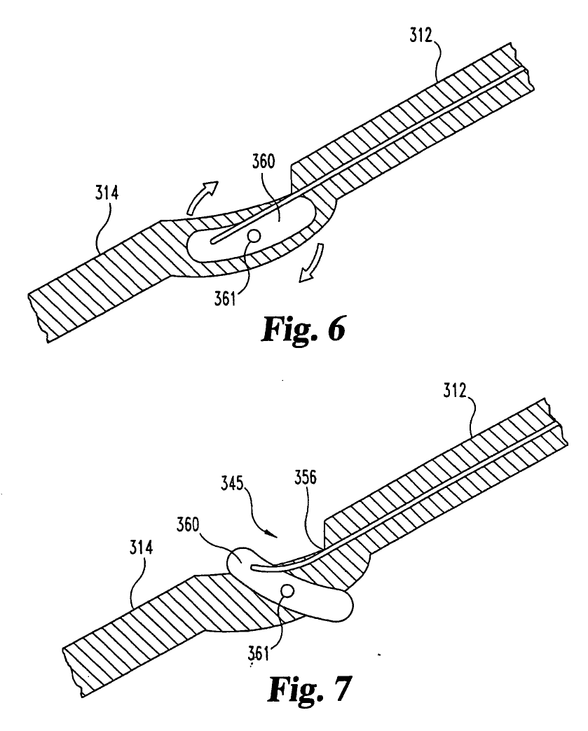

FIG. 5 is a cross-sectional view of suturing device 310. Foot 360 can be controlled or deployed by lever 362 using cable 367 that extends from foot 360 through an interior channel or lumen in member 312 to connect to one end of lever 360. Foot 360 can, but need not, include a biasing element such as a spring 365 to either urge foot to reside in the first deployed position or the second non-deployed position. As illustrated, spring 365 urges lever 362 and foot 360 to a non-deployed position. It will be understood that cable 367 can also extend along side of member 312 without requiring a separate channel or lumen.

FIG. 6 illustrates intermediate member 316 with foot 360 in a non-deployed position. Movement of lever 362 causes foot 360 to rotate about pin 361 to a deployed position to support the vascular tissue from inside the vessel lumen as illustrated in FIG. 7. It should be apparent to those skilled in the art that a variety of mechanical interconnections may be used to translate the movement of lever 362 into rotation of foot 360 about end 361. In addition to the cable connection 367, it may be possible to employ a rack which engages a cog on foot 360 so that when the rack is displaced longitudinally the foot 360 rotates from the non-deployed to the deployed condition.

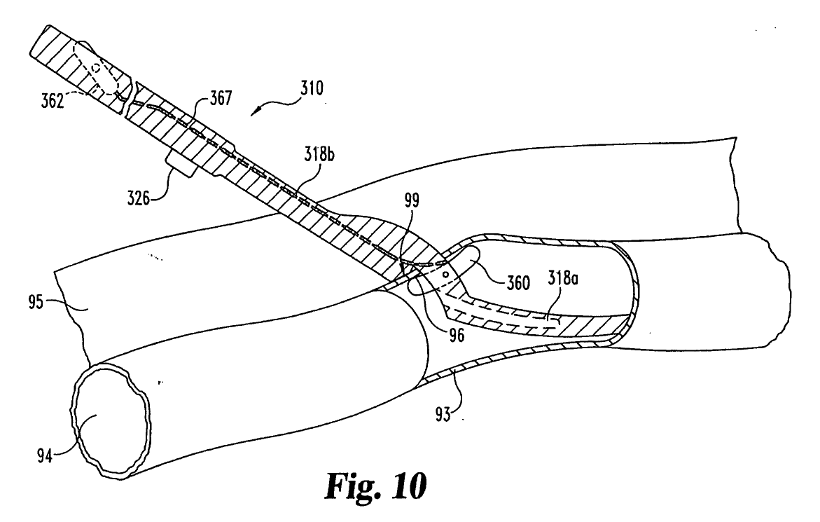

FIGS.8 through 10 illustrate use of suturing device 310 in accordance with the present invention. Distal end 314 of suturing device 310 can be initially inserted into a vascular vessel 94. For this procedure it will be understood that device 310 includes the blood return line 325, opening 352 and stop 356 for device 10 that operate in cooperation to ensure accurate placement of the suturing device in the patient's vascular vessel. However for the purposes of clarity, these elements have not been illustrated in the present drawing.

Once distal member 314 and at least a portion of intermediate member 316 have been inserted into the vascular vessel as desired, foot 360 can be deployed. In certain embodiments, intermediate member 316 is inserted sufficiently deeply (distally) into the lumen 93 of vessel 94 so that the foot 360 is completely enveloped within the interior of lumen 93 to facilitate deployment of foot 360. This positioning allows facile deployment of foot 360 such at the tissue engaging surfaces 368 and 369 of ends 363 and 364, respectively readily support and bear against inner or distal vascular tissue adjacent the wound 96 without catching on vascular tissue surrounding the wound. Deployment lever 362 on proximal member 312 can be pivotally rotated urging the cable 367 to deploy foot 360. Once foot 360 has been fully deployed, the suturing device 310 can be withdrawn in the proximal direction, if necessary, to allow the upper surfaces 368 and 369 of the foot to support and even exert slight pressure on the internal tissue of the vascular vessel as shown in FIG. 9. Thereafter, a needle 318a can be advanced though a needle channel 324 in proximal member 312 using needle pusher 326 to pierce vascular tissue adjacent the wound 96 at a first puncture site 97 and into receptacle 322. Needle 318a can remain within receptacle 322 during further manipulation of the device during wound closure.

Referring now to FIG. 10, suturing device 310 can be rotated about its longitudinal axis to a second suturing position. In general, the same procedure as described above can be employed for the present embodiment. However, slight modifications of the procedure can facilitate the use of the present device. First distal member 314 need not be removed or completely removed from the lumen 94 of vessel 93. However, it may be preferable to retract foot 360 to the non-deployed position prior to rotation of the device to the second suturing position. This can reduce the risk that foot 360 will exert unnecessary force and/or torque on the vascular tissue, which could cause injury such as tears or over extension of the tissue. Once the device is repositioned at the second suture position, foot 360 can be re-deployed using lever 362 as described above.

As illustrated in FIG. 10, a second needle 318b can be advanced through the needle channel 324 using needle pusher 326 to pierce vascular tissue at a second puncture site 99. Second needle 318b can be further advanced into receptacle 322.

After the first and second needles 318a and 318b have pulled suture material through the vascular tissue, the suturing device 310 can be withdrawn from the vessel. This results in a suture path that extends in a distal direction through the wound in the vessel to a first suture site in the vascular tissue; out through the first suture site in a proximal direction; across the wound on the proximal side of the tissue to a second suture site; then in a distal direction through the second suture site back into the lumen of the vessel and from there back out in the proximal direction through the wound as illustrated in FIG. 11. If needles 318a and 318b have a single piece of suture material 320 connected to their proximal end; the suture material illustrated in FIG. 11 is a single strand with two ends extending through the puncture wound 96. If needles 318a and 318b have separate suture material connected to their proximal end, there would be a different configuration. Furthermore, if the suture needles are configured to capture a needle retention element the needle capture element as illustrated and described in co-pending U.S. patent application ______, filed on (and listed on attorney docket 7528-3), the suture material will take still a different path. In any event, accurate positioning of the suturing device 310 is enabled with the deployable foot 360.

Once the device 310 is sufficiently removed or completely removed, the needles 318a, 318b and the attached suture material can be gathered in an appropriate fashion depending upon the number of individual lengths of suture material. When desired, the needles can be separated from the suture material. The suture material is gathered to gather to effect wound closure. A suture knot can be tied by hand. A loosely tied knot can be advanced to the external surface of the vessel using a knot pusher. Alternatively, a knot box can be used to form a knot which is then advanced to the external surface of the vessel. Examples of suitable knot boxes are illustrated and described in WO 01/19258, which is incorporated herein by reference.

Alternatively, one or more knot replacement technologies such can be used to secure closure of the wound using the suture material. Consequently, the suture knot or suture securing devices can be positioned across the proximal side of the wound to effect closure.

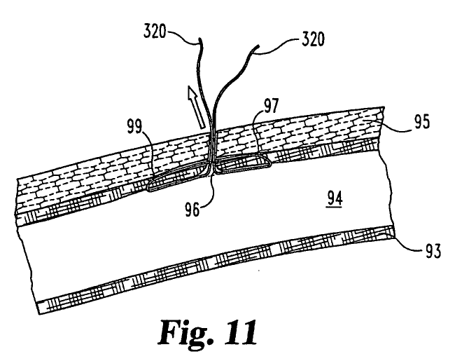

FIG. 12 is a perspective view of a suture securing device 102 for use in the present invention. Suture securing device 102 is described and illustrated in US Patent Publication No. 2004/0102809 which is incorporated herein by reference. In use, device 102 can secure ends of one, two, three or more lengths of suture material. Two lengths of suture material 104a and 104b are illustrated with device 102. The lengths of suture material are threaded into the flexible elements 105a and 105b which are then locked or fixed together securing the suture material therein.

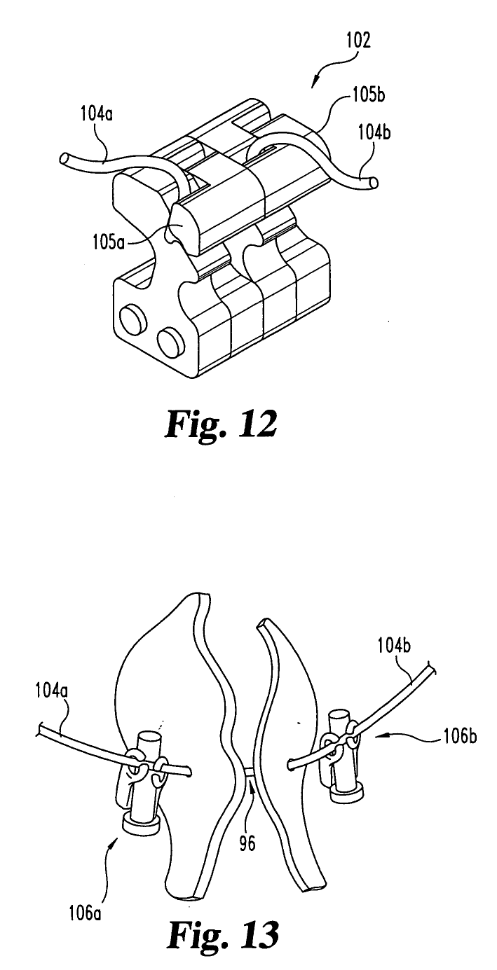

FIG. 13 is another embodiment of suture securing devices 106a and 106b for use in the present invention. Devices 106a and 106b are described in US Patent Publication No. 2003/0229377 which is incorporated herein by reference in its entirety. Devices 106a and 106b cooperate by separately clipping onto a selected length of suture material 104a or 104b which have previously pulled taut to close wound or complete the surgical procedure. The devices prevent the suture material from regressing back through the sutured tissue.

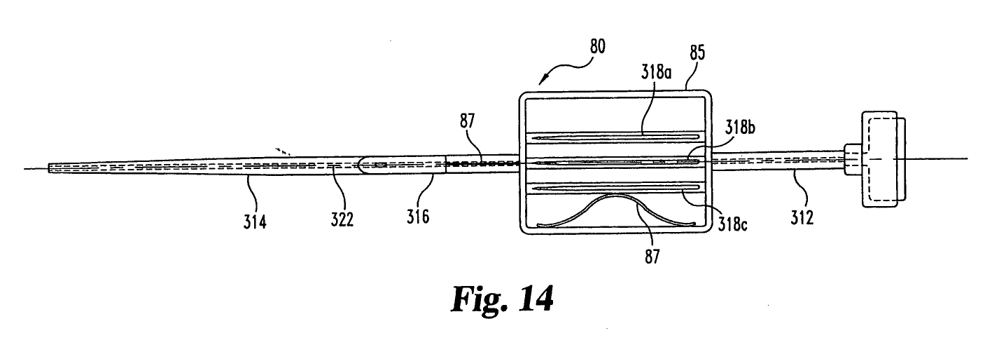

FIG. 14 shows still other embodiment in which the receptacle 322 is sized to receive one two or more needles 318a, 318b, 318c, which can be retained without any further modifications. Receptacle 322 includes one or more needle capture elements, for example, one or more plugs 123 or pierceable material elements or projection(s) that can be pierced by needles advanced into receptacle 322. In this embodiment, needle 318a is captured within the receptacle reducing the risk of dislocation during manipulation of the suturing device. Examples of suitable needle capturing elements are illustrated and described in co-pending U.S. patent application No. ______, filed on (filed under Attorney Docket No. 7528-3), which is incorporated herein by reference.

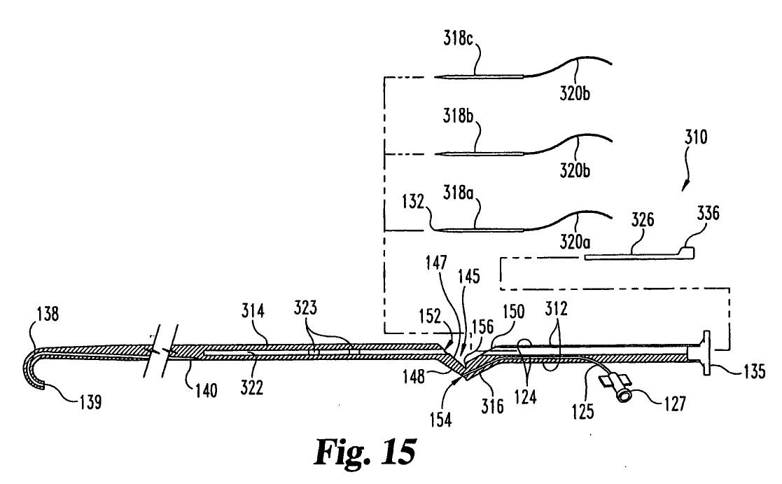

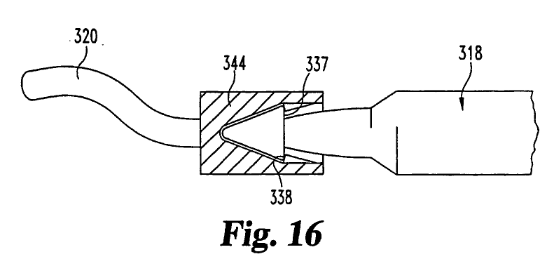

As shown in FIGS. 15 and 16, one or more needle engaging fittings attached to suture material can be positioned within receptacle 322. In this embodiment, needles 318a, 318b, 318c , . . . can each include a recessed surface 337 suitable for engaging with tabs 338 on fittings 344 in the receptacle 322. Fittings 44 may be attached to suture material 320, also at least partially contained within receptacle 322. Examples of suitable fittings and needles are illustrated and described in co-pending U.S. patent application No. ______, filed on (under Attorney Docket No. 48011-2), which is incorporated herein by reference. (Note: FIGS. 1 and 3 from 48011-2 should be included here renumbered as FIG. 15 and 16.

The present invention provides a variety of means, devices and methods for closing wounds in tissue and is particularly but not exclusively suitable for vascular tissue. It will be understood that the present invention contemplates modifications as would occur to those skilled in the art without departing from the spirit of the present invention. In addition, the various structures, elements, and procedural steps or stages have been described with reference to specified embodiments and devices. Each of the individual or a combination of the structures, elements, and procedural steps or stages are contemplated to be combinable with each of the other embodiments and devices described herein and as such are contemplated to be within the scope of the present invention.

All publications, patents, and patent applications cited in this specification are herein incorporated by reference as if each individual publication, patent, or patent application was specifically and individually indicated to be incorporated by reference and set forth in its entirety herein. Further, any theory of operation, proof, or finding stated herein is meant to further enhance understanding of the present invention and is not intended to make the scope of the present invention dependent upon such theory, proof, or finding.

What is claimed is:

1. A suturing device for suturing an opening in a vascular vessel, said device comprising:
a proximal member including an elongate body having a needle channel therethrough sized to receive at least one needle;
a distal member configured to be inserted within a lumen of a vascular vessel, said distal member having a receptacle located therein;
an intermediate member disposed between the proximal member and the distal member, said intermediate member defining a tissue receiving area and having a first opening providing a passageway to the channel and a second opening providing a passageway into the receptacle; and
a foot pivotally mounted on the intermediate member about a pivot point.

2. The device of claim 1 comprising a lever operably connected to the foot, wherein the lever is mounted on the proximal member.

3. The device of claim 1 wherein a distal portion of the intermediate member is configured to be inserted within the vascular vessel whereby upon articulation the foot engages with vascular tissue on a distal side of the vessel.

5. The device of claim 1 wherein the intermediate member includes a concave wall portion configured to nest the foot therein.

4. The device of claim 1 wherein the foot is provided in a first position to facilitate insertion through the opening in the vascular vessel.

5. The device of claim wherein the foot is configured to articulate to a second position to support the distal side of the vascular tissue.

6. The device of claim 1 comprising a biasing element operable to urge the foot into a first position.

7. The device of claim 1 wherein the foot is symmetric about the pivot point.

8. The device of claim 1 wherein the foot is asymmetric about the pivot point.

7. The device of claim 6 comprising a lever operable to pivot the foot from a first position to a second position.

8. The device of claim 1 wherein the proximal member comprises a needle cartridge having two or more slots for needles.

9. The device of claim 8, wherein the cartridge is slidably mounted on the proximal member to sequentially align each of the two or more slots with the needle channel.

10. The device of claim 1 comprising a needle pusher slidably mounted in alignment with the channel to advance a needle along the channel in a direction toward the first opening in the connecting member.

11. The device of claim 1 wherein the receptacle is sized to receive two or more needles therein.

12. The device of claim 12 wherein the proximal member and the distal member define a substantially linear longitudinal axis and the intermediate member deviates from said linear longitudinal axis.

13. The device of claim 12 wherein the intermediate member comprise a convex surface and a concave surface.

14. The device of claim 12 wherein the first opening and the second opening are aligned along the linear longitudinal axis.

15. The device of claim 12 wherein the distal member comprises a distal end that deviates from the linear longitudinal axis.

16. The device of claim 1 configured to provide an essentially linear needle pathway from the channel to the receptacle.

17. The device of claim 16 wherein the essentially linear needle pathway extends through the tissue receiving area.

18. The device of claim 16 wherein the foot is positioned adjacent the tissue receiving area.

19. The device of claim 16 wherein the foot is operable to be positioned distal to the tissue receiving area.

20. The device of claim 1 wherein the intermediate member comprises means for positioning the device within the lumen of the vascular vessel.

21. The device of claim 1 wherein the distal member is flexible.

22. The device of claim 24 wherein the intermediate member is rigid.

23. A method of suturing an opening in a vascular vessel, said method comprising:
inserting a suturing device through the opening in the vascular vessel, said suturing device comprising a proximal member having a needle channel therein; a distal member configured to be inserted into the lumen of the vascular vessel, the distal member having a receptacle therein sized to receive at least one needle therein; an intermediate member disposed between the proximal and distal members, the intermediate member having a first opening providing a passageway to the needle channel and a second opening providing a second passageway into the receptacle and wherein the intermediate member is angled or curved to offset the needle channel and the receptacle from the opening in the vascular vessel; and a foot pivotally mounted on the intermediate member;
deploying the foot to contact vascular tissue adjacent the opening;
advancing a first needle through the needle channel to pierce a portion of vascular tissue at a first suture site adjacent the opening in the vascular vessel and into the receptacle in the distal member.

24. The method of claim 23 comprising repositioning the suturing device to a second suturing site.

25. The method of claim 26 wherein the needle carries a length of suture material.

26. The method of claim 28 wherein the step of advancing said needle draws a length of suture material distally through a portion of vascular tissue adjacent the opening in the vascular vessel.

27. The method of claim 24 comprising retracting the foot to a first position prior to repositioning the suturing device.

28. The method of claim 26 wherein said deploying comprises pivoting the foot from a first position adjacent the tissue receiving area to a second position distal to the tissue receiving area.

29. The method of claim 26 wherein said deploying comprises deploying the foot after said inserting step.

30. The method of claim 27 comprising advancing a second needle carrying a second length of suture material through the needle channel to pierce tissue at the second suture site.

31. The method of claim 28 comprising withdrawing the suturing device from the vascular vessel.

32. The method of claim 29 wherein said withdrawing comprises drawing the first and second lengths of suture material through the opening in a proximal direction.

33. The method of claim 30 comprising securing the first length of suture material to the second length of suture material.

34. The method of claim 32 wherein said securing comprises tying the first and second portions of suture together.

35. The method of claim 32 wherein said securing comprises clamping the first and second portions of suture together with a suture securing device.

36. The method of claim 30 wherein the first length of suture material and the second length are two ends of a single length of suture material.
